(19) United States
(12) Plant Patent Application Publication
(10) Pub. No.: US 2008/0229458 P1
(43) Pub. Date: Sep. 18, 2008

(54) Apple tree named 'Masonova'

(75) Inventors: Charles G. Embree, Port WIlliams (CA); A. David Crowe, Wolfville (CA)

Correspondence Address: KLARQUIST SPARKMAN, LLP, 121 SW SALMON STREET, SUITE 1600, PORTLAND, OR, 97204, US

(73) Assignee: Her Majesty The Queen in Right of Canada, as represented by The Minister of Agriculture and Agr

(21) Appl. No.: 11/725,127

(22) Filed: Mar. 15, 2007

(51) Int. Cl. A01H 5/00

(52) U.S. Cl. PLT/170

(57) ABSTRACT

A new apple variety distinguished by resistance to apple scab; good cold storage ability; desirable eating characteristics including soft and fine textured ivory colored flesh with some green and red venation surrounding the core, and flesh exhibiting absent to very weak browning after cutting; and the skin has solid, complete red coloration.

### CROSS REFERENCE TO RELATED APPLICATIONS

None

### STATEMENT REGARDING FEDERALLY SPONSORED RESEARCH OR DEVELOPMENT

None

### LATIN NAME OF THE GENUS AND SPECIES OF THE PLANT CLAIMED

Malus domestica Borkh.

### VARIETY DENOMINATION

‘Masonova’

### BACKGROUND OF THE INVENTION

The present invention relates to a new and distinct variety of apple tree named ‘Masonova’. The new tree ‘Masonova’ (experimental designation S47-24-42) resulted from a planned hybridization programme and is a selection from a cross between the pollen parent ‘Empire’ and the seed parent ‘S21-42-69’ in 1974. The cultivar ‘Empire,’ which is a cross between McIntosh and Red Delicious, was released by the Cornell Agricultural Experiment Station in Geneva, N.Y., in 1966. ‘Empire’ is a cultivar that gained wide commercial acceptance for a number of reasons including productivity under a wide range of planting systems, harvest during the gap between McIntosh and Red Delicious; resistance to preharvest fruit drop and storage scald, low susceptibility to fire blight, and fruit quality with respect to color, firmness, and storage. The resulting tree was selected when growing in a cultivated area in Nova Scotia, Canada.

More specifically, pollen was collected from the cultivar ‘Empire’, stored and dried for use in the traditional crossing method. At the full bloom stage, the emasculated flowers of ‘S21-42-69’ were pollinated with the dried pollen from ‘Empire’. Seeds were collected from successfully pollinated fruit in the fall of 1974. In 1978, trees were planted in the seeding evaluation field block of Agriculture and Agri-Food Canada (AAFC) in Kentville, Nova Scotia. Fruit was evaluated from 1984 to 1988 and re-propagation for field evaluations was done in 1991.

The selection of ‘Masonova’ took place in 1989. Selection criteria for ‘Masonova’ included scab resistance, combined with the desirable eating quality traits of ‘Empire’ and solid, complete red coloration of the fruit.

Trees of ‘Masonova’ were compared to ‘Novaspy’ and ‘Liberty’ apple varieties, referred to herein as the “reference varieties.” ‘Novaspy’ originated at AAFC as a cross between Nova Easygro×(Red Spy×Golden Delicious), while ‘Liberty’ originated at the New York State Agricultural Experiment Station, Geneva, N.Y., as a cross between Macoun×Purdue 54-12.

The comparative tests and trials for ‘Masonova’ were conducted in 2003 and 2004 by AAFC. The fruit and tree characteristics were evaluated on trees planted in 1992 in a cooperative scab-resistant cultivar trial set up by AAFC in cooperation with grower E.W. Peillat Starr's Point, Nova Scotia. In recent years, the orchard has been managed by J.W. Mason & Sons, Ltd. The trial consisted of four trees of each variety grafted on ‘Budogovsky 490’ rootstock. They were planted twelve feet apart in rows which were spaced at eighteen feet.

Asexual reproduction shows that the distinguishing characteristics of ‘Masonova’ are established and transmitted through succeeding asexual propagations.

### SUMMARY OF THE INVENTION

The ‘Masonova’ variety is distinguished from other apple varieties due to the following unique combination of characteristics: resistance to apple scab; good cold storage ability; desirable eating characteristics including soft and fine textured ivory colored flesh with some green and red venation surrounding the core, and flesh exhibiting absent to very weak browning after cutting; the skin has solid, complete red coloration. Tree shape is upright with a spur type growth habit. Fruit shape is flat, globose with a pronounced eye basin and stalk cavity. Petal shape is orbicular.

Asexual reproduction of this new variety by grafting and budding onto ‘Budogovsky 490’ rootstock shows that the foregoing and other characteristics come true to form, are firmly fixed, and are established and transmitted through succeeding propagations.

The original Masonova crosses were conducted in 1974. Seeds produced from these crosses were planted in 1978. The following detailed description concerns the original tree (S47-24-42), selected in 1989, and progeny first asexually propagated in 1991.

The original tree and progeny have been observed growing in a cultivated area in Nova Scotia, Canada. Certain characteristics of this variety, such as growth and color, may change with changing environmental conditions (e.g., light, temperature, moisture, nutrient availability, or other factors). Color descriptions and other terminology are used in accordance with their ordinary dictionary descriptions, unless the context clearly indicates otherwise. Color designations are made with reference to The Royal Horticultural Society (RHS) Color Chart. All color characteristics were determined using the 1986 version of The Royal Horticultural Society (RHS) color charts and measured characteristics were based on ten plant measurements. It should be understood that the colors may vary, depending on factors such as growing and lighting conditions.

### BRIEF DESCRIPTION OF THE DRAWINGS

The ‘Masonova’ apple is illustrated by the accompanying color photographs, in which:

FIG. 1 shows ‘Masonova’ flower buds and flowers;

FIG. 2 shows one-year old ‘Masonova’ shoots;

FIG. 3 shows a close-up view of a one-year old ‘Masonova’ shoot;

FIG. 4 shows a stem-view of two ‘Masonova’ apples;

FIG. 5 shows cross-sectional views of ‘Masonova’ apples; and

FIG. 6 shows a close-up view of a cross-section of a ‘Masonova’ apple;

FIG. 7A and 7B show one side of ‘Masonova’ leaves;

FIG. 8 shows ‘Masonova’ tree during winter;

FIG. 9 shows “Masonova’ tree bark;

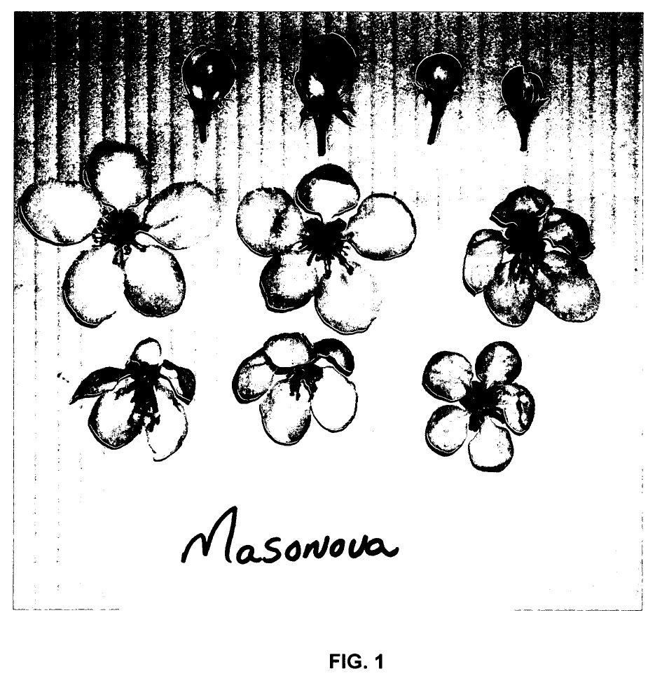

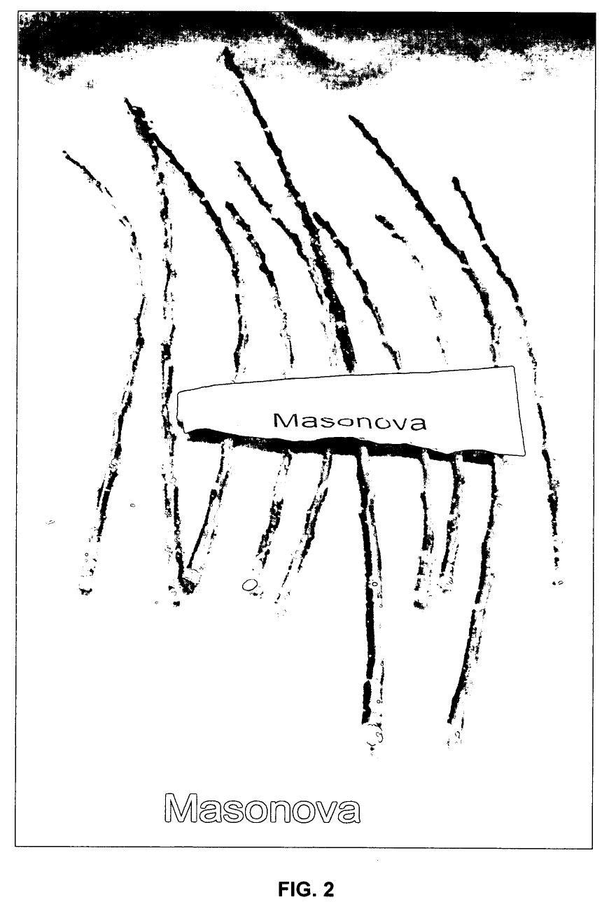

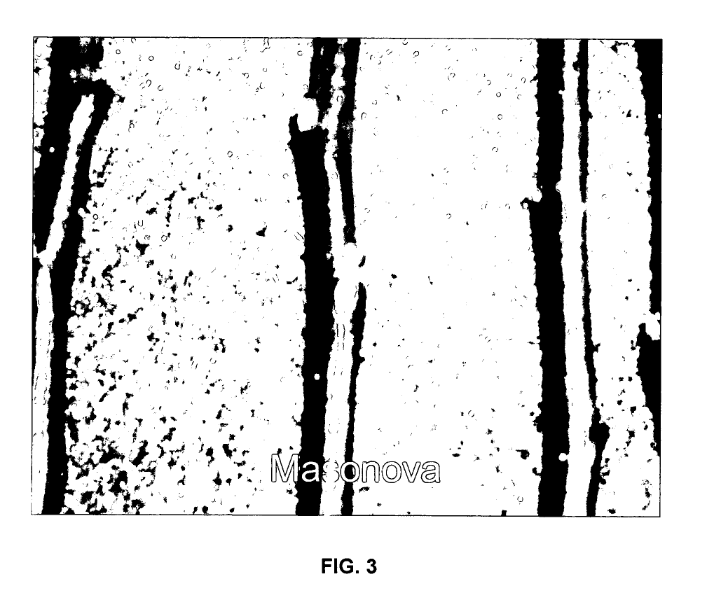

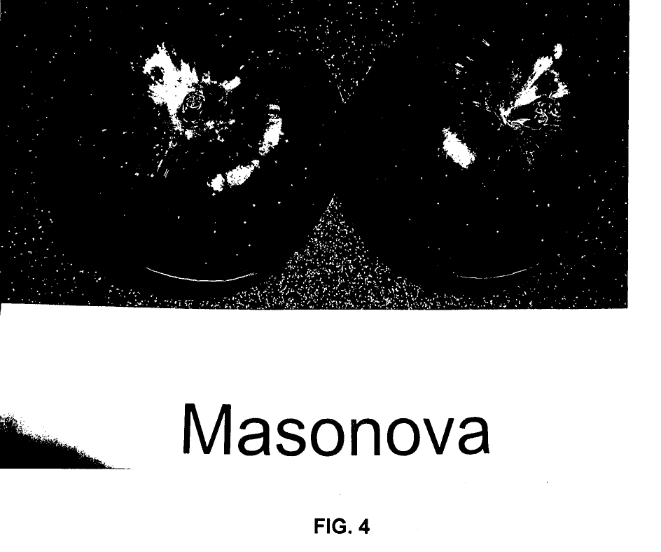

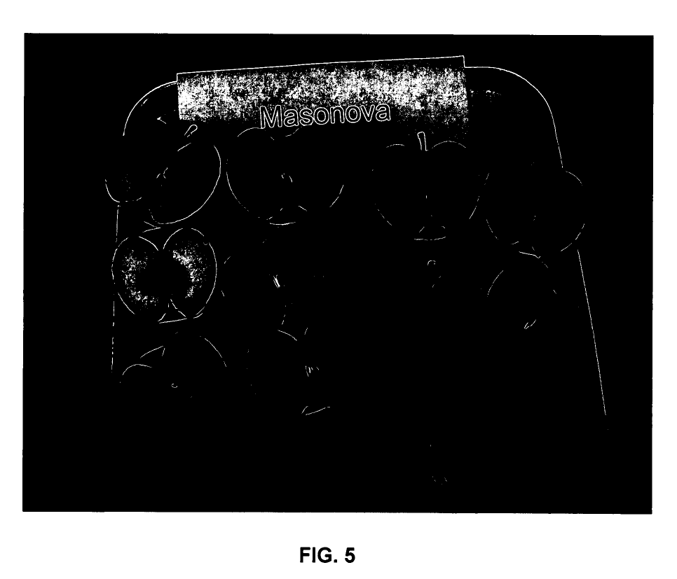

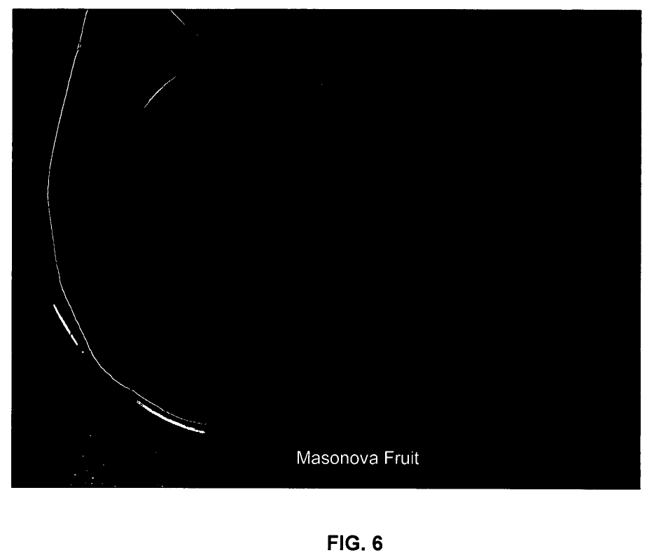

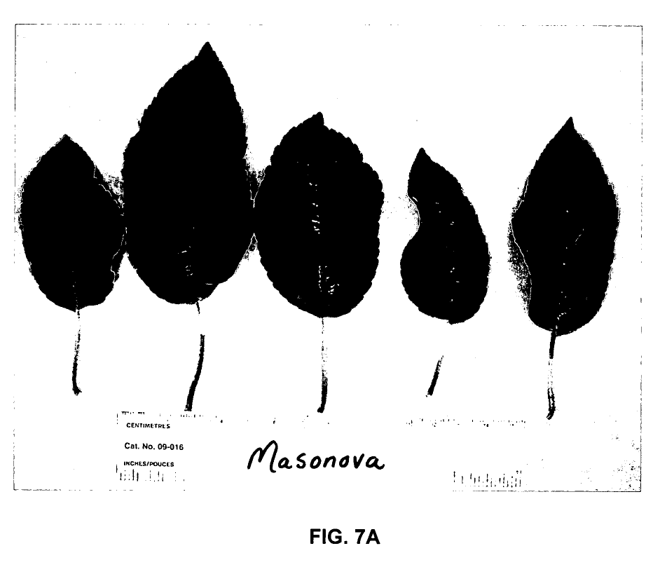

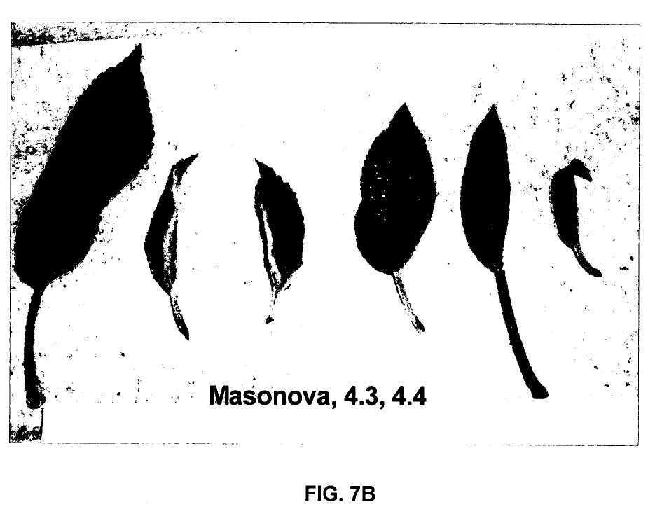

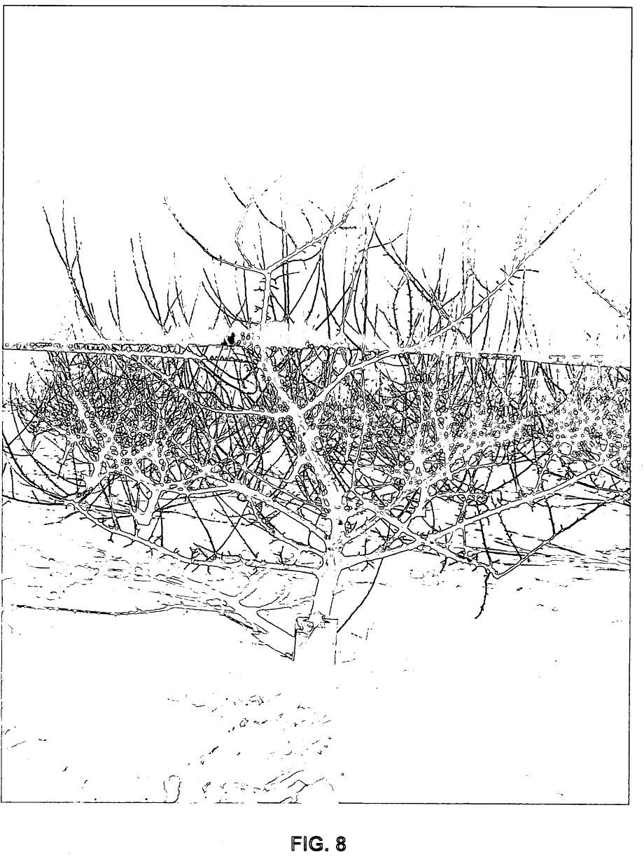

The colors of an illustration of this type may vary with lighting and other conditions, therefore, color characteristics of this new variety should be determined with reference to the observations described herein, rather than from these illustrations alone.

### DETAILED DESCRIPTION

‘Masonova’ is a mid-season dessert apple variety having a moderately good fruit setting with winter hardiness to Canadian zone 5b. ‘Masonova’ is resistant to scab (Iventuria inaequalis) and may be suitable for farming using organic growing techniques. The preferred method of propagation is budding onto rootstock liners in a nursery, although other suitable asexual growing methods may be used.

### BOTANICAL

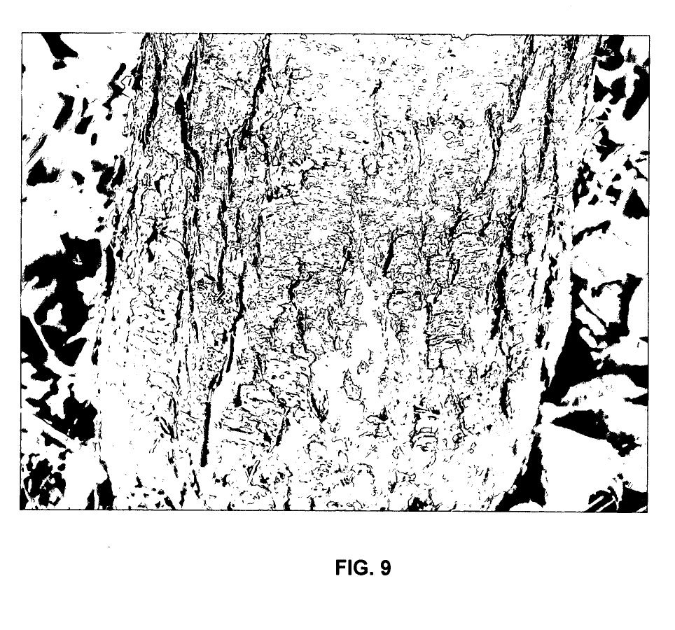

The following detailed description of the ‘Masonova’ variety is based on observations of asexually reproduced progeny. The observed progeny are trees which were 12 years of age and growing on ‘Budogovsky 490’ rootstock in Nova Scotia, Canada.
Scientific Name: Malus domestica Borkh. ‘Masonova’.
Parentage:
   Seed parent.—‘S21-42-69’.
   Pollen parent.—‘Empire’.
Tree:
   Vigor.—moderate.
   Overall shape.—upright.
   Height.—About 14-18 ft.
   Width.—Overall spread of about 12 ft.
   Caliper.—TCA cm²=100 cm².
   Trunk bark texture.—thin, flaky, with longitudinal cracks (see FIG. 9.).
   Trunk bark color.—brown (RHS 166A).
   Patches or other markings.—lenticels present on trunk (horizontal) whitish in color.
   Primary branches.—moderate to strong branches; the angle of the fruit bearing branches is less than 90° and the major fruit load is predominantly borne on spurs.
   Branch color.—One-year old branches are brown-red (RHS 187A) in color, while two-year old branches are brown (RHS 166A) in color.
   Branch pubescence.—One year old shoots have absent to very sparse pubescence on the upper half.
   Thickness.—Dormant one-year old shoot have mean thickness of 5.3 mm, with a standard deviation of 0.72.
   New growth bark.—One year old shoots have moderately shiny bark and are brown (RHS 187A) in color.
   Branch lenticels.—One year old shoots have few lenticels on the upper half; the lenticels have a low density, approximately 4-6 per square inch; typical examples of which measured about 1.27 inch in diameter; beige brown (RHS 165D) in color.
   Internodes.—Average internode length is about 24.3 mm on a one-year old shoot.
   Bearing.—Heavy bearing tree.
   Hardiness.—At least Zones 5b (Canada).
   Disease resistance.—Field immune to scab (Iventuria inaequalis).
Leaves:
   Size.—small to medium.
   Length.—About 49.6 mm to about 87.7 mm, averaging about 66.9 mm.
   Width.—mean 35.8 mm, with a standard deviation of 4.85.
   Margin.—crenate.
   Tip shape.—acuminate.
   Pubescence.—The length, width, thickness and other measurements were obtained from observations of ten typical leaves in 2003.
   Shoot tip leaves.—concave in cross section, with sparse pubescence on the upper side. The lower side of the shoot tip leaves is green with no secondary coloration.
Flowers:
   Size.—single flowers, 4.3-5.5 cm, typical flower measuring about 5.0 cm across.
   Color.—Unopened bud: medium pink to dark pink (RHS 65A). Opened flower: light blue pink on inner surface of petals (RHS 62B); blue pink on outer surface of petals (RHS 65B).
   Petals.—5 petals per flower; orbicular in shape that touch an overlap; mean width 18.3 mm, with a standard deviation of 1.16; mean length 20.8 mm, with a standard deviation of 1.23.
   Bloom season.—seasonal flower development classified as very late; full bloom observed on Jun. 8, 2003.
Fruit: (Observations from a sample of typical fruit in 2003.)
   Size.—medium; mean diameter of 7.29 cm, with a standard deviation of 0.40.
   Form.—asymmetrical and flat globose; no ribbing; eye is medium in size with an open aperture.
   Stalk cavity.—pronounced; broad and medium in depth; mean width 3.7 cm, with a standard deviation of 0.14.
   Eye Basin.—deep and medium in width; mean depth 6.44 mm, with a standard deviation of 0.99; about 3.0 cm wide.
   Stem.—medium in length and thickness; typical average observed in 2003, about 18 mm long and 1.9 mm in diameter; green (RHS 162B) in color.
   Locules.—closed.
   Skin.—surface is smooth with no bloom and some waxiness.
   Lenticels.—slightly prominent and small in size.
   Color.—General color effect: Dark Red Ground color: creamy white (RHS 18C) Overcolor: very high amount of dark red in a striped blush pattern (RHS 187B) Russetting: none.
   Fruit properties at maturity (based on 10 fruit tested in 2006). Firmness: moderate, about 10.13-12.85 kg, averaging about 10.52 kg. (Fruit in storage for 10 weeks) Texture: fine. Soluble solids: About 13.0%, averaging about 13.0%. Flavor: Mild, sweet, sub-acid Juiciness: moderate Flesh color: ivory with some green and red venation surrounding the core (RHS 158C). Flesh browning: absent to very weak one hour after being cut. Aroma: strong, ripe apple.
   Core.—Core line is absent to very weakly distinct; Median bundle area shape; typical in circular about 36 mm long and about 36 mm wide.
   Seed.—Brown and normal in shape at maturity; about 2-3 seeds per cell; normal shaped; about 8.26 mm long and about 4.33 mm wide; brown (RHS 177A) in color.
   Fruit production.—First picking date in October in 2003 was about 10th, and last picking date was about same; average production is 400-500 lbs. of fruit per tree.
   Storage.—Fruit remains fresh at room temperature for 10 days, and can be stored up to 180 days in air storage at 3-4° C.
   Usage.—dessert apple, juice or sauce.

The following is a comparison of some characteristics of the ‘Masonova’ apple variety to the reference varieties ‘Novaspy’ and ‘Liberty’.

‘Masonova’ produces moderately vigorous, upright trees with moderate to strong branches; the angle of the fruit bearing branches is +/−90° and the major fruit load is predominantly borne on spurs. By contrast, the branches of ‘Liberty’ have a spreading habit and those of ‘Novaspy’ are weeping. Fruit bearing occurs on both the spurs and shoots for ‘Novaspy’. ‘Masonova’ has stronger branches and wider leaves than the reference varieties. Dormant one year old shoots of ‘Masonova’ are thicker, less pubescent and have fewer lenticels than those of either reference variety.

Seasonal flower development is classified as very late for ‘Masonova’ while it is mid-season for ‘Novaspy’ and mid-season to late for ‘Liberty’. ‘Masonova’ flowers are single with orbicular shaped petals that touch an overlap, while petal shape is ovate to oblong for ‘Novaspy’ and ovate for ‘Liberty’. The color of the flower buds of ‘Masonova’ is a lighter blue pink than those of the reference varieties.

‘Masonova’ produces medium sized fruit that are asymmetrical and flat globose in shape with a pronounced eye basin and stock cavity whereas ‘Novaspy’ produces large fruit which is truncate conical in shape. In comparison to the fruit of the reference varieties, ‘Masonova’ has a larger eye aperture, deeper eye basin, wider stalk cavity and more over color of the skin than the reference varieties.

The color of the fruit flesh for ‘Masonova’ is ivory with some green and red venation surrounding the core while it is cream for Novaspy’ and white for ‘Liberty’. Browning of the fruit flesh is absent to very weak for ‘Masonova’ one hour after being cut, moderate for ‘Novaspy’ and strong for ‘Liberty’. The fruit of ‘Masonova’ has softer flesh than the reference varieties. The texture of the flesh is fine for ‘Masonova’ while it is coarse for ‘Novaspy.’

Table 1 quantifies some differences between ‘Masonova’ and the reference varieties.

TABLE 1
Comparison table for ‘Masonova’

| | | |
|---|---|---|
| ‘Masonova’ | ‘Novaspy’* | ‘Liberty’* |

Thickness of dormant one-year old shoot (mm)

| | | | |
|---|---|---|---|
| mean | 5.3 | 3.8 | 4.8 |
| std. deviation | 0.72 | 0.48 | 0.59 |

Leaf width (mm)

| | | | |
|---|---|---|---|
| mean | 35.8 | 49.9 | 52.8 |
| std. deviation | 4.85 | 6.42 | 5.75 |

Petal width (mm)

| | | | |
|---|---|---|---|
| mean | 18.3 | 16.6 | 15.6 |
| std. deviation | 1.16 | 0.70 | 0.52 |

Color offlower buds (RHS)

| | | |
|---|---|---|
| 65A | 63B | 63B |

Color of petals (RHS)

| | | | |
|---|---|---|---|
| inner side | 62B | 62C | 62B |
| outer side | 65B | 63C | 65A |

Fruit diameter (cm)

| | | | |
|---|---|---|---|
| mean | 7.29 | 7.78 | 7.07 |
| std. deviation | 0.40 | 0.45 | 0.46 |

Depth of fruit eye basin (mm)

| | | | |
|---|---|---|---|
| mean | 6.44 | 5.83 | 4.51 |
| std. deviation | 0.99 | 1.12 | 0.69 |

Width of stock cavity (cm)

| | | | |
|---|---|---|---|
| mean | 3.7 | 3.4 | 2.9 |
| std. deviation | 0.14 | 0.37 | 0.18 |

*reference
variety

We claim:

1. A new and distinct variety of apple tree, substantially as herein shown and described.
